(19) United States
(12) Patent Application Publication
(10) Pub. No.: US 2004/0064051 A1
(43) Pub. Date: Apr. 1, 2004

(54) Ultrasound transducer coupling apparatus

(76) Inventors: Roger J. Talish, Hillsborough, NJ (US); Donald E. Krompasick, Bethlehem, PA (US); Ian Cutts, Memphis, TN (US)

Correspondence Address: David M. Carter, Esq., Carter, DeLuca, Farrell & Schmidt, LLP, Suite 225, 445 Broad Hollow Road, Melville, NY, 11747, US

(21) Appl. No.: 10/261,281

(22) Filed: Sep. 30, 2002

(51) Int. Cl. A61B 8/14

(52) U.S. Cl. 600/459

(57) ABSTRACT

An apparatus is provided which includes a substantially flat base defining a pocket insert configured and dimensioned for receiving the treatment head module and a hydrogel in alignment with the treatment head module for preventing the attenuation of the ultrasonic waves propagated from the treatment head module during ultrasonic treatment. A portion of the apparatus which comes into contact with the patient is provided with an adhesive covered by a peel-off backing. During use, the peel-off backing is removed to expose the adhesive and the apparatus is affixed to the patient. The treatment head module is then inserted within the pocket insert. The pocket insert securely maintains the treatment head module in position without over compressing soft or injured tissue. In another embodiment thereof, the apparatus includes a hydrogel having an adhesive on opposing sides and peel-off backings covering each adhesive. During use, the peel-off backings are removed to expose each adhesive. One of the adhesives is used to affix the apparatus to the patient, while the other adhesive is used to affix the treatment head module thereon.

### BACKGROUND OF THE INVENTION

1. Field of the Invention

The present invention relates to an apparatus for mounting an ultrasound transducer. More particularly, the present invention relates to an apparatus for mounting an ultrasound transducer which includes a skin adhesive side, a side for mounting the transducer head module, and an ultrasound conductor provided in between the two sides.

2. Description of the Related Art

The use of ultrasound to therapeutically treat musculoskeletal injuries is known. Impinging ultrasonic energy having appropriate parameters, e.g., frequency, pulse repetition, and amplitude, for suitable periods of time and at a proper external location adjacent to a bone injury has been determined to accelerate the natural healing of, for example, bone breaks and fractures and to treat osteoporosis. For patients with reduced healing capacity, such as elderly persons with osteoporosis, ultrasonic therapy may promote healing of bone injuries which would otherwise require prosthetic replacement or leave the patient permanently disabled.

U.S. Pat. Nos. 5,003,965 and 5,186,162, both to Talish and Lifshey (“Talish '965” and “Talish '162”, respectively) and U.S. Pat. No. 5,520,612 to Winder et al. describe an ultrasonic delivery system where the RF generator and transducer are both part of a modular applicator unit that is placed at the skin location. The signals controlling the duration of ultrasonic pulses and the pulse repetition frequency are generated apart from the applicator unit. Talish '965 and Talish '162 also describe fixture apparatus for attaching the applicator unit so that the operative surface is adjacent the skin location. In Talish '965 and Talish '162, the skin is surrounded by a cast, while in U.S. Pat. No. 5,211,160 to Talish and Lifshey (“Talish '160”) fixture apparatus is described for mounting on uncovered body parts (i.e., without a cast or other medical wrapping).

In many instances, the patient receiving ultrasound therapy treatment is mobile. A transducer head module may be mounted on the patient remote from a stationary ultrasound generator, or portable ultrasound generating apparatus may be carried by the patient as disclosed, for example, in U.S. Pat. Nos. 5,556,372 and 6,165,144, both to Talish et al. The transducer head module is therefore increasingly more prone to external impacts which may move the module or adversely affect the treatment efficiency. Thus, while the systems described in the prior art disclose typical therapeutic ultrasound methods and apparatus, they do not disclose a method and apparatus for mounting an ultrasound transducer without the existence of a cast which prevents the transducer head module from shifting or moving.

Further, conventional transducer mounting apparatus is often bulky and cumbersome to the patient, especially when the apparatus is located on a portion of the patient's body which is typically covered by clothing. Thus, a need exists for a mounting apparatus which resists being shifted from external impacts, has a low profile when mounted on the patient to facilitate comfort to the patient while the apparatus is mounted on a location under clothing, and does not compress soft or injured tissue.

Further still, conventional transducer mounting apparatus require application of a gel or a placement of a gel pad or pillow to the treatment site prior to providing the transducer head module. Gel is slippery and difficult to maintain at desired location while gel pads or pillows need to be held in place prior to positioning the transducer head module. Thus, a need exists for a mounting apparatus which resists being shifted from external impacts and contains a built-in ultrasound conductive pad or hydrogel.

Additionally, conventional transducer mounting apparatus typically require straps and bandages for securing the transducer head module to the treatment site. Thus, a need exists for an apparatus which resists being shifted from external impacts and does not require straps and/or bandages for securing the transducer head module to the treatment site.

### SUMMARY

It is an object of the present invention to provide an apparatus having a low profile for easily mounting an ultrasound transducer treatment head module which overcomes these and other disadvantages of the prior art. In an embodiment thereof, the apparatus includes a substantially flat base defining a pocket insert configured and dimensioned for receiving the treatment head module and a hydrogel in alignment with the treatment head module for preventing the attenuation of the ultrasonic waves propagated from the treatment head module during ultrasonic treatment. A portion of the apparatus which comes into contact with the patient is provided with an adhesive covered by a peel-off backing. During use, the peel-off backing is removed to expose the adhesive and the apparatus is affixed to the patient. The treatment head module is then inserted within the pocket insert. The pocket insert securely maintains the treatment head module in position without over compressing soft or injured tissue.

In another embodiment thereof, the apparatus includes a hydrogel having an adhesive on opposing sides and peel-off backings covering each adhesive. During use, the peel-off backings are removed to expose each adhesive. One of the adhesives is used to affix the apparatus to the patient, while the other adhesive is used to affix the treatment head module thereon. The adhesive securely maintains the treatment head module in position without over compressing soft or injured tissue.

These and other objects, features and advantages of the present invention will become apparent from the following detailed description of illustrative embodiments, which is to be read in connection with the accompanying drawings.

### BRIEF DESCRIPTION OF THE FIGURES

For a better understanding of the invention, reference is made to the following description of exemplary embodiments, and to the accompanying drawings, wherein:

FIG. 1 is a perspective view of an apparatus according to an embodiment of the present invention;

FIG. 2 is a top view of the apparatus of FIG. 1;

FIG. 3 is a bottom view of the apparatus of FIG. 1;

FIG. 4 is a top view of the apparatus of FIG. 1 showing an ultrasound transducer treatment head module being connected thereto;

FIG. 5 is a side view of the apparatus of FIG. 1 affixed to a patient and having the ultrasound transducer treatment head module connected thereto;

FIG. 6 is a perspective view of an apparatus according to another embodiment of the present invention;

FIG. 7 is a top view of the apparatus of FIG. 6 showing a hydrogel provided within the apparatus; and

FIG. 8 is a side view of the apparatus of FIG. 6 affixed to a patient and having an ultrasound transducer treatment head module attached thereto.

### DETAILED DESCRIPTION

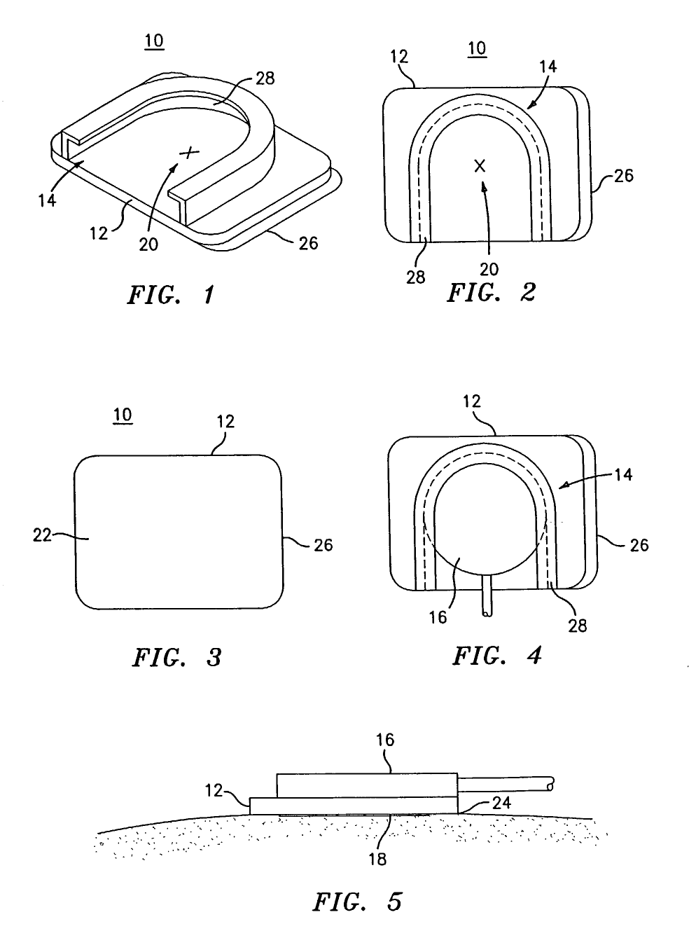

Referring now to the drawings in detail, and initially to FIGS. 1 to 3, an apparatus is shown according to an embodiment of the present invention and designated generally by reference numeral 10. The apparatus 10 includes a substantially flat, durable base 12 defining a pocket insert 14 configured and dimensioned for receiving an ultrasonic transducer treatment head module 16 (see FIG. 4). The treatment head module 16 is of the type shown and described in U.S. Pat. No. 6,165,144 to Talish et al., the contents of which are incorporated herein by reference. The base 12 is preferably manufactured from plastic, cardboard, or other biocompatible material, and measures approximately 3.5 cm by 3.0 cm.

The apparatus 10 further includes a hydrogel 18 which is positioned beneath the base 12. A central portion of the base 12 includes a marking 20 for aiding in locating the treatment site by defining a specific location in radiographically sighting the treatment site.

A portion of one side 22 of the apparatus 10 which comes into contact with the patient is provided with an adhesive 24 covered by a peel-off backing 26. Preferably, adhesive 24 is a skin-grade adhesive, such as OPSITE™ adhesive available commercially from Smith & Nephew, Inc. To affix the apparatus 10 to the patient, the peel-off backing 26 is removed by grabbing the portion of the peel-off backing 26 extending from the base 12. Upon removing the peel-off backing 26, the adhesive 24 is exposed and the apparatus 10 can then be affixed to the patient.

The treatment head module 16 is then inserted within a concave track 28 of the pocket insert 14 and slidably received therein as shown by FIG. 4. It is provided that the height of the track 28 is narrower than the portion of the treatment head module 16 that is slidably received by the track 28. Therefore, the portion of the treatment head module 16 that is received by the track 28 is press fit therein. Hence, the apparatus 10 strongly maintains the treatment head module 16, including during external impacts. The treatment head module 16 is connected to a main operating unit (not shown), as shown and described by U.S. Pat. No. 5,556,372 to Talish et al. which is hereby incorporated by reference, by a connector 30.

As shown in FIG. 5, when the treatment head module 16 is connected to the apparatus 10, the hydrogel 18 aligns with the treatment head module 16 for substantially preventing the attenuation of the ultrasonic waves propagated from the treatment head module 16. It is provided that after treatment, the treatment head module 16 is removed from the pocket insert 14 by sliding the treatment head module 16 out from the track 28. The apparatus 10 is then removed from the patient by pulling the apparatus 10 off the patient.

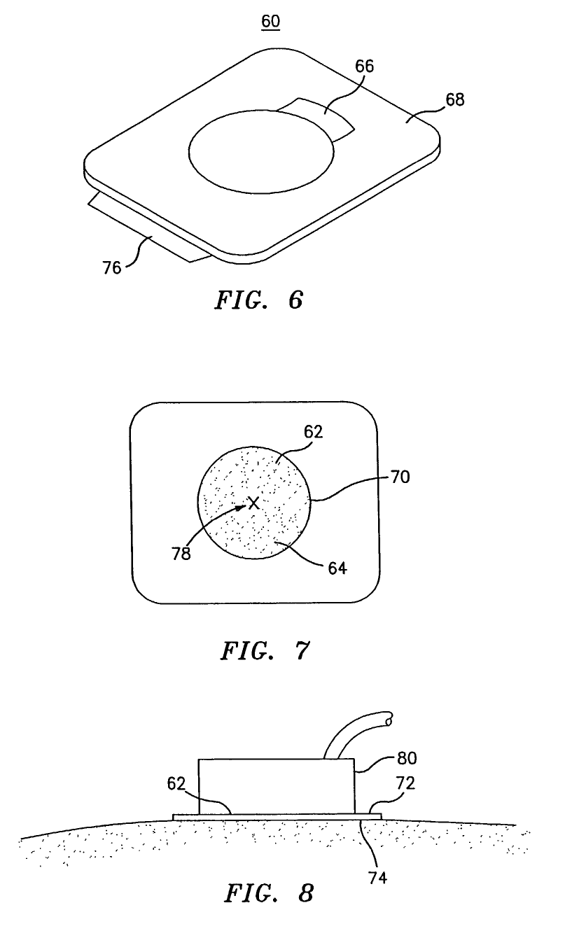

With reference to FIGS. 6 and 7, there is shown an apparatus in accordance with another embodiment of the present invention and designated generally by reference numeral 60. The apparatus 60 preferably measures approximately 3.0 cm by 3.0 cm and includes a hydrogel 62. The hydrogel 62 includes an adhesive 64 and a peel-off backing 66 on a top side 68. The hydrogel 62 is positioned within a central opening 70 defined by a base or an adhesive-backed carrier 72. The central opening 70 is configured and dimensioned for receiving the hydrogel 62 therein.

One side of the adhesive-backed carrier 72 has an adhesive 74 covered by a peel-off backing 76. A central portion of the hydrogel 62 includes a marking 78 for aiding in locating the treatment site.

During use, the peel-off backing 76 is removed from the apparatus 60 to expose the adhesive 74 disposed on the adhesive-backed carrier 72. The apparatus 60 is then affixed to the patient. The peel-off backing 66 on the top side 68 is then removed from the apparatus 60 to expose the adhesive 64 disposed on the hydrogel 62 (see FIG. 7). An ultrasonic transducer treatment head module 80 (see FIG. 8) is then affixed to the adhesive.

The treatment head module 80 is connected to a main operating unit. As shown by FIG. 8, it is recommended that the treatment head module 80 is aligned with the central portion of the hydrogel 62 for substantially preventing the attenuation of the ultrasonic waves propagated from the treatment head module 80. It is provided that after treatment, the treatment head module 80 is removed from the adhesive 64 by pulling the treatment head module 80 from the adhesive 64. The apparatus 60 is then removed from the patient by pulling the apparatus 60 off the patient.

The embodiments described herein provide a simple to use ultrasonic transducer coupling apparatus which prevents or reduces the compression of soft or injured tissue and maintains the treatment head module in position even during external impacts. Further, the apparatus described herein enables one to couple the treatment head module without using straps or bandages. The apparatus further enables one to radiograpically sight or pinpoint the treatment site prior to treatment. These and other advantages as evident to one ordinarily skilled in the art are provided by the apparatus of the present invention.

Although the illustrative embodiments of the present invention have been described herein with reference to the accompanying drawings, it is to be understood that the invention is not limited to those precise embodiments, and that various other changes and modifications may be affected therein by one skilled in the art without departing from the scope or spirit of the invention. All such changes and modifications are intended to be included within the scope of the invention as defined by the appended claims.

We claim:

1. A transducer coupling apparatus comprising:
a base having a first surface with a biocompatible adhesive thereon for removably securing said base to a skin surface of a patient, said base having a second surface configured to operatively maintain an ultrasound transducer in ultrasound coupling engagement with the patient.

2. The apparatus according to claim 1, further comprising a hydrogel positioned between the adhesive and the second surface to facilitate ultrasound coupling with the patient.

3. The apparatus according to claim 2, wherein one of the base and the hydrogel include a marking for aiding in sighting a treatment site.

4. The apparatus according to claim 3, wherein the marking is in the hydrogel.

5. The apparatus according to claim 1, wherein the second surface includes a pocket insert configured and dimensioned for receiving the ultrasound transducer head module.

6. The apparatus according to claim 5, wherein the pocket insert includes a track for slidably receiving at least a portion of the ultrasound transducer head module.

7. The apparatus according to claim 6, wherein at least one dimension of the track is narrower than the portion of the ultrasound transducer head module which is received by the track, wherein the portion of the ultrasound transducer head module is press fit within the track.

8. The apparatus according to claim 1, wherein the second surface includes an adhesive disposed on the surface to attach at least a portion of the ultrasound transducer.

9. The apparatus according to claim 6, wherein the adhesive disposed on the surface is covered by a backing material.

10. The apparatus according to claim 1, wherein the second surface is substantially flat.

11. An apparatus for mounting an ultrasound transducer head module to a patient, the apparatus comprising:
a base having a pocket insert configured and dimensioned for receiving at least a portion of the ultrasound transducer head module;
a hydrogel positioned against the base opposite the pocket insert; and
an adhesive disposed in proximity to the hydrogel, the adhesive being covered by a peel-off backing, wherein during use the peel-off backing is removed from the apparatus to expose the adhesive and the apparatus is affixed to the patient, and wherein the portion of the ultrasound transducer head module is inserted within the pocket insert and connected to the base.

12. The apparatus according to claim 11, wherein the base includes a marking for aiding in sighting a treatment site.

13. The apparatus according to claim 11, wherein the pocket insert includes a track for slidably receiving the portion of the ultrasound transducer head module.

14. The apparatus according to claim 13, wherein at least one dimension of the track is narrower than the portion of the ultrasound transducer head module which is received by the track, wherein the portion of the ultrasound transducer head module is press fit within the track.

15. The apparatus according to claim 11, wherein the base is substantially flat.

16. An apparatus for mounting an ultrasound transducer head module to a patient, the apparatus comprising:
a hydrogel having an adhesive disposed on a top surface, the adhesive being covered by a peel-off backing; and
a base having means for receiving the hydrogel and an adhesive covered by a peel-off backing, wherein the base adhesive affixes the base to the patient, and wherein the adhesive on the top surface of the hydrogel mounts the ultrasound transducer head module.

17. The apparatus according to claim 16, wherein the top surface of the hydrogel includes a marking for aiding in sighting a treatment site.

18. The apparatus according to claim 16, wherein the means for receiving the hydrogel includes an opening configured and dimensioned for receiving the hydrogel therein.

19. The apparatus according to claim 16, wherein the base is substantially flat.

20. A method for mounting an ultrasound transducer head module to a patient, the method comprising the steps of:
providing a base having a pocket insert configured and dimensioned for receiving at least a portion of the ultrasound transducer head module;
positioning a hydrogel against the base opposite the pocket insert; and
disposing an adhesive in proximity to the hydrogel, the adhesive being covered by a peel-off backing, wherein during use the peel-off backing is removed from the apparatus to expose the adhesive and the apparatus is affixed to the patient, and wherein the portion of the ultrasound transducer head module is inserted within the pocket insert and connected to the base.

21. The method according to claim 20, wherein the pocket insert includes a track for slidably receiving the portion of the ultrasound transducer head module.

22. The method according to claim 21, wherein at least one dimension of the track is narrower than the portion of the ultrasound transducer head module which is received by the track, wherein the portion of the ultrasound transducer head module is press fit within the track.

23. The method according to claim 20, wherein the base is substantially flat.

24. An apparatus having a double adhesive for mounting an ultrasound transducer head module to a patient, the apparatus comprising:
a top surface having a first adhesive disposed thereon and covered by a peel-off backing; and
a bottom surface opposite the top surface having a second adhesive covered by a peel-off backing, wherein the bottom surface adhesive affixes the bottom surface to the patient, and wherein the top surface adhesive mounts the ultrasound transducer head module to the top surface.

25. The apparatus according to claim 24, wherein the top surface is a surface of a hydrogel.

26. The apparatus according to claim 24, wherein the bottom surface is a surface of a substantially flat base.
